(19) United States
(12) Patent Application Publication
(10) Pub. No.: US 2016/0178598 A1
(43) Pub. Date: Jun. 23, 2016

(54) ENHANCING RESERVOIR FLUID ANALYSIS USING PARTITIONING COEFFICIENTS

(71) Applicant: HALLIBURTON ENERGY SERVICES, INC., Houston (TX)

(72) Inventors: Michael T. Pelletier, Houston, TX (US); Matthew Dennis Rowe, Lafayette, LA (US); Dale E. Jamison, Humble, TX (US); Christopher Michael Jones, Houston, TX (US)

(21) Appl. No.: 14/389,464

(22) PCT Filed: Jun. 20, 2013
(86) PCT No.: PCT/US2013/046771

§ 371 Date: Sep. 30, 2014

(51) Int. Cl. G01N 33/28; G01N 21/31

(52) U.S. Cl. CPC G01N 33/2823; G01N 21/31

(57) ABSTRACT

Methods and systems of analyzing a reservoir fluid based on surface measurements of fluids circulated in a wellbore are provided. A fluid (e.g., a drilling fluid) with known properties is circulated within a wellbore where it is exposed to a reservoir fluid. A gas extractor is used to analyze a sample of the gas phase of the circulated fluid to determine the composition of the gas phase. Partitioning coefficients are used to calculate the composition of the solid, aqueous, organic, and gas phases of the circulated fluid. The composition of the fluid before and after circulation through the wellbore is compared to determine the composition of the reservoir fluids.

### BACKGROUND

As oil well drilling becomes increasingly complex, it is desirable to collect and analyze information relating to the formation. One way to collect this information is by analyzing a circulated fluid, such as the drilling fluid. A drilling fluid or “mud” is a specially designed fluid that is circulated in a wellbore or borehole as the wellbore is being drilled in a subterranean formation to facilitate the drilling operation. The various functions of a drilling fluid include removing drill cuttings from the wellbore, cooling and lubricating the drill bit, aiding in support of the drill pipe and drill bit, and providing a hydrostatic head to maintain the integrity of the wellbore walls and prevent well blowouts.

Properties of the drilling fluid are typically monitored during drilling operations. For instance, it is often desirable to accurately measure hydrocarbon gas concentrations of the drilling fluid as it leaves the wellbore. The level of the hydrocarbon gas in the drilling fluid may affect how the well is to be drilled as well as the safety of the drilling rig and personnel involved. Moreover, the concentration of hydrocarbon gases and other components present in the drilling fluid may be indicative of the characteristics of the formation being drilled and the drilling environment.

Accordingly, the analysis of drilling fluids and the changes they undergo during drilling operations is an important factor in optimizing the drilling operations and may be important to the methods of drilling as well as the efficiency of the drilling operations. Consequently, during drilling, completion and testing of a wellbore, it is desirable to obtain analytical measurements of the fluids that are returned to the surface from the wellbore.

One method for collecting and analyzing the drilling fluid involves submerging a rotor within a vessel into the drilling fluid as the drilling fluid exits the wellbore. The drilling fluid is agitated as it enters into and exits out of the vessel and some of the gasses dissolved therein evaporate and escape the confines of the fluid. These vaporized gases are then collected and processed by analytical methods to determine the presence and levels of hydrocarbons and other components in the drilling fluid.

However, as a drilling fluid is exposed to a subterranean reservoir containing gases, those gases partition into different fluids present in the wellbore depending on various characteristics of the reservoir. When those fluids are circulated back to the surface, the gas content is often measured by extracting those gases from the fluid. Conventional gas extraction methods generally do not distinguish how the gases (or how much of them) partitioned into different fluids. For example, the existing methods do not measure residual saturation amounts in the aqueous phase, nor do they account for the respective amounts of a component in the oil and aqueous phases.

Indeed, certain conventional techniques of surface wellsite analysis may result in undesirable phase transitions. Previous endeavors to solve the problem attempted to account for this problem by providing complicated procedures for sampling the fluid in the wellbore itself. However, downhole analysis often requires stopping the circulation in the wellbore, which can lead to several problems. Stopping the circulation can cause economic hardships by delaying production. It can also cause damage as the contents of the wellbore settle.

It is thus desirable to provide methods and systems that can more accurately measure and analyze fluid samples taken from a reservoir to determine the characteristics of the reservoir from which the fluid sample was taken.

### BRIEF DESCRIPTION OF THE DRAWINGS

These drawings illustrate certain aspects of some of the embodiments of the present invention, and should not be used to limit or define the invention.

FIG. 1 illustrates an exemplary wellbore and the flow of a circulated fluid within the wellbore.

FIG. 2 illustrates one example of a system for extracting gas that can be used to practice the present invention.

### DETAILED DESCRIPTION

The present disclosure provides a more complete analysis of a sample of a circulated fluid to obtain more accurate data regarding the nature and composition of a subterranean reservoir to which the circulated fluid has been exposed. Existing methods rely on one or more correction coefficients to estimate the efficiency with which components are converted to gas. While existing gas analysis methods force gas out of the circulated fluid sample and measure the composition of only that gaseous phase, the methods of the present disclosure will evaluate the flow and composition of all phases of the mud sample. This includes the gas, aqueous, organic, and solid phases. The teachings of the present disclosure directly account for the efficiency with which components are converted to gas by calculating or measuring the amount of a component in each phase, eliminating the need for an estimated correction coefficient.

The methods and systems of the present disclosure utilize partitioning coefficients to quantify the transitions between the four phases of the circulated fluid sample. Certain partitioning coefficients describe the transition between the solid phase and each of the other three phases. Other partitioning coefficients describe the transition between the aqueous phase and the organic or gas phase. Finally, partitioning coefficients describe the transition between the organic and gas phases. These partitioning coefficients can be used to describe the transition between two phases in both directions. By using the appropriate partitioning coefficients, it is possible to determine the composition of one phase by measuring the composition of a different phase (e.g., the gas phase).

The methods and systems of the present disclosure may be used in a wellbore disposed in a subterranean formation. A wellbore may be created so as to extend into a reservoir located in the subterranean formation. In one embodiment, a casing may be disposed within the wellbore and cement may be introduced between the casing and the wellbore walls in order to hold the casing in place and prevent the migration of fluids between the casing and the wellbore walls. A tubing string may be disposed within the casing. In an embodiment, the tubing string may be jointed tubing, coiled tubing, or any other type of tubing suitable for use in a subterranean well environment. Suitable types of tubing and an appropriate choice of tubing diameter and thickness may be known to one skilled in the art, considering factors such as well depth, pressure, temperature, chemical environment, and suitability for its intended use.

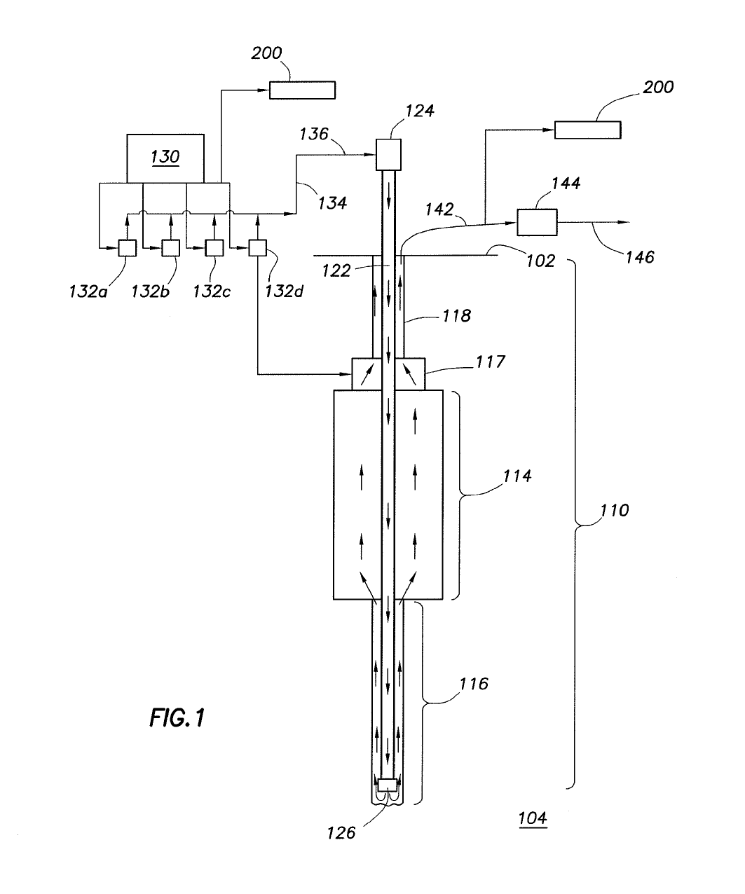

FIG. 1 illustrates one example of a typical drilling operation in which the present disclosure can be used. In the exemplary drilling operation, a wellbore 110 is drilled from the drill floor 102 to a subterranean formation 104 containing a reservoir. The wellbore may include cased hole 114 and open hole 116. In the cased hole 114, the wellbore 110 is sealed off from the subterranean formation 104 with metal casing, cement, or other means. In the open hole 116, the wellbore 110 is exposed to the subterranean formation 104 and fluids may flow between the wellbore 110 and the subterranean formation 104. A blowout preventer (BOP) stack 117 may be disposed above the cased hole 114. A riser 118 may connect the blowout preventer to the surface. A drill string 122 may be disposed within the wellbore 110. A top drive 124 may rotate the drill string 124 to turn a bit 126 located at the bottom of the drill string 122.

The methods and systems of the present disclosure may be used with any fluid that is circulated in the wellbore 110. During drilling operations, drilling fluid (or “mud”) is typically circulated. The drilling fluid or mud may comprise any base fluid, including but not limited to water, oil, synthetic oil and/or synthetic fluid. In certain embodiments, the drilling fluid may further comprise solids suspended in the base fluid. A non-aqueous based mud may contains oil or synthetic fluid as a continuous phase and may also contain water dispersed in the continuous phase by emulsification so that there is no distinct layer of water in the fluid. Such dispersed water in oil is generally referred to as an invert emulsion or water-in-oil emulsion. A number of additives may be included in such drilling fluids and invert emulsions to enhance certain properties of the fluid. Such additives may include, for example, emulsifiers, weighting agents, fluid-loss additives or fluid-loss control agents, viscosifiers or viscosity control agents, and alkali.

The density of the drilling mud is maintained in order to control the hydrostatic pressure that the mud exerts at the bottom of the well. If the mud is too light, formation fluids, which are at higher pressures than the hydrostatic pressure developed by the drilling mud, can enter the wellbore and flow uncontrolled to the surface, possibly causing a blowout. If the mud is too heavy, then the hydrostatic pressure exerted at the bottom of the wellbore can reduce the rate at which the drill bit will drill the hole. Additionally, excessive fluid weights can fracture the formation causing serious wellbore failures. A person of skill in the art with the benefit of this disclosure will know how to use the appropriate additives to control the weight of the mud.

As shown in FIG. 1, the drilling mud is circulated in the wellbore 110 through the drill string 122. Initially, the drilling mud is pumped to the drill string 122 from an active pit system 130. Several booster pumps 132a-d may be used to help move the drilling mud. The drilling mud may be pumped through a stand pipe 134 and a kelly hose 136 to the top of the drill string 122. The drilling mud is pumped from through the drill string 122 where it exits the drill string 122 through the bit 126. The drilling mud then flows back up to the surface through the annular space between the drill string 122 and the wellbore 110. When it reaches the surface, the drilling mud flows through a flow out line 142. It passes through a cleaning system 144 before entering a return line 146 that may return the drilling mud to the active pit system 130.

According to an embodiment of the present disclosure, the physical characteristics of the mud are determined before the mud is introduced to the wellbore. For example, the initial flow volume and composition of all four phases (solid, aqueous, organic, and gas) of a drilling mud are measured. Based on the measured characteristics of the mud, the specific parameters of the mud can be calculated. For example, a set of partitioning coefficients for all four phases in the mud sample (solid, aqueous, organic, and gas) can be generated. The partitioning coefficients are based on phase equilibrium, which may be corrected for pressure, temperature history, particle effects, and/or other conditions. The phase equilibrium model choice depends on its suitability to pressure and temperature. It is also possible for several phase equilibrium models to be used to properly determine the partitioning coefficients at different points in the circulation system. These can be modified for non-Newtonian fluid phase behavior and the addition of solid particles crossing between phases.

Therefore, mud with known properties is introduced into the wellbore. As the mud circulates within the wellbore, it interacts with the formation fluids present in the reservoir. The concentration of components of the mud (e.g., hydrocarbons) changes depending on, among other things, the composition of the formation fluid in the reservoir.

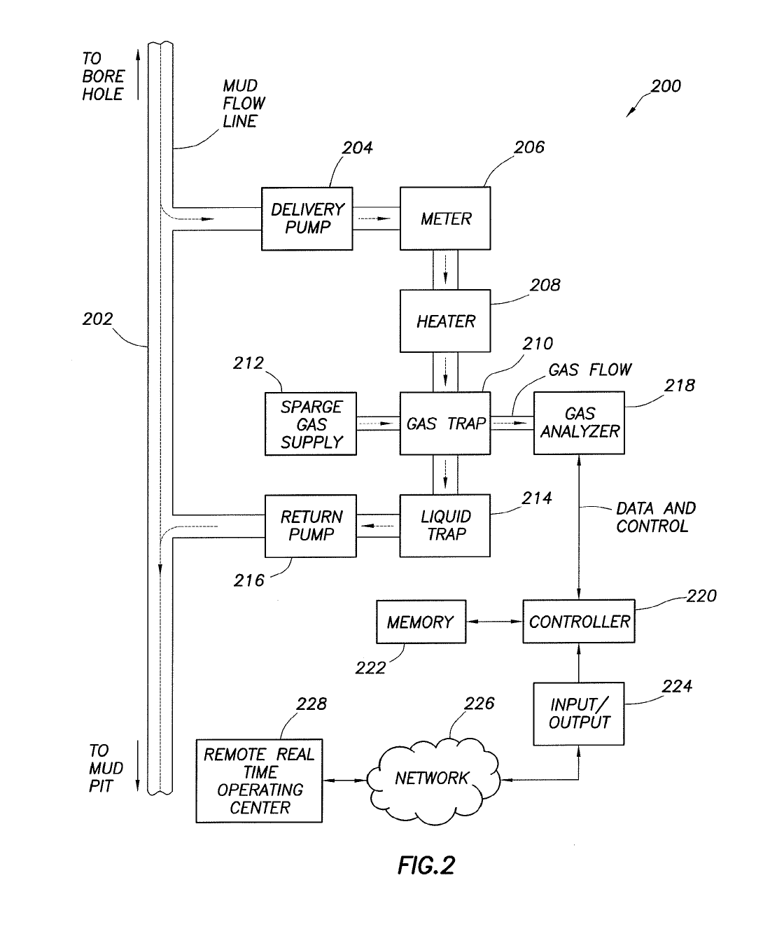

When the mud is returned to the surface, a gas sample is extracted and analyzed in a gas extraction system. Any suitable gas extractor may be used with the methods and systems of the present disclosure. One example of a suitable gas extractor is described by U.S. Patent Application Publication No. 2011/0219853. Another example of a suitable gas extractor is the EAGLE™ available from Halliburton Energy Services, Inc. Another example of a suitable gas extractor is the Constant Volume Extractor (CVE) gas system available from Halliburton Energy Services, Inc. An exemplary gas extractor is system 200, which is illustrated as the block diagram of FIG. 2.

In system 200, a delivery pump 204 pumps drilling mud from the mud flow line 202. The delivery pump 204 produces a constant reliable volume of drilling mud from the mud flow line 202 into the system. The delivery pump 204 includes a peristaltic pump.

A meter 206 measures the volume of drilling mud that has been extracted from the mud flow line 202 by the delivery pump 204. A heater 208 heats the mud from the meter 206 to a constant mud temperature. The constant mud temperature is selected to liberate hydrocarbon gases, such as alkanes (C1 methane through the hydrocarbon range to C12 dodecane), aromatics such as benzene and toluene, and olefins such as ethene (acetylene) and mercaptans. The heater heats the mud to a temperature of approximately (e.g., within 10 percent of) 80 degrees Centigrade.

The mud from the heater 208 is sent to a gas trap 210, which extracts gas from the drilling mud. A sparge gas supply 212 is coupled to the gas trap to introduce an inert gas, such as nitrogen, into the gas trap. The gas trap 210 produces a gas output and a liquid output. The liquid output is sent to a liquid trap 214. A return pump 216 pumps the liquid out of the liquid trap 214 and back into the mud flow line 202. The liquid trap 214 is part of the gas trap 210.

The gas output of the gas trap 210 is sent to a gas analyzer 218, which analyzes the components of the gas output. This gas output is the gas sample. A carrier gas may be added to the gas sample at the point of gas extraction. A carrier gas can be any gas and serves to help pump the gas sample to the gas analyzer. Suitable carrier gases will be known to a person of skill in the art with the benefit of this disclosure and can include atmospheric gas, nitrogen, or helium. The gas analyzer may account for the presence of the carrier gas.

Gas analyzer 218 may be any equipment known in the art that is capable of analyzing a gas phase sample. For example, in some embodiments, gas analyzer 218 uses gas spectroscopy. In other embodiments, gas analyzer 218 may include a hydrocarbon analyzer. Other analyzers may include mass spectrometers, laser spectrometers, and infrared spectrometers. In other embodiments, the gas analyzer may include solid state chemical detectors. The gas analyzer 218 reports its results to a controller 220, which also receives data (not shown) from the meter 206.

The controller 220 is a special purpose computer programmed to perform the functions described herein. The controller 220 is coupled to a memory 222. The memory 222 contains the programs to be executed as the controller 220 performs its functions as well as constants and variables used to perform those functions. The controller 220 may be coupled to one or more input/output devices 224, such as a keyboard, a mouse, a monitor or display, a speaker, a microphone, or a network interface. The controller 220 may also be coupled to a network 226, such as a local area network or the Internet, either directly or through one or more of the input/output devices 224. The controller 220 may also be coupled to a remote real time operating center 228 through the input/output devices 224 and the network 226, allowing the remote real time operating center 228 to control and receive data from the controller 220.

The controller 220 receives data from and controls other elements of the system 200 including: displaying and/or controlling the delivery pump 204 flow rate; displaying and/or controlling the heater 208 temperature; displaying and/or controlling the return pump 216 flow rate; displaying and/or controlling the blow back rate; displaying the density, flow rate, and temperature of the drilling mud measured by the meter 206; displaying the gas trap 210 temperature; displaying and/or controlling the gas trap 210 rotation rate; displaying and/or controlling the liquid trap 214 temperature.

The methods and systems of the present disclosure use the gas extractor to measure the amount of a component (e.g., a particular hydrocarbon) in the gas phase. The partitioning coefficients can then be used to calculate an amount of the measured component in each of the other phases of the mud.

The appropriate partitioning coefficients depend on a number of factors that will be recognized by a person of skill in the art with the benefit of this disclosure. These factors include the temperature, the specific pressures, the phases, the boundary layers, the presence of surfactants, and the presence of other additives. The partitioning coefficients may be determined in a variety of ways depending on the source of the data. In one embodiment, they can be calculated using mathematical models. In another embodiment, they can be determined by performing a laboratory analysis on theoretical fluids. In a preferred embodiment, they can be determined from wellsite data. A person of skill in the art would understand how to determine the appropriate partitioning coefficients.

The methods and systems of the present disclosure may be used to analyze and monitor mud samples throughout the entire system, or at just one portion of the system (e.g., just at the gas extractor interface). This creates a profile of a particular component in each phase of the drilling mud. The difference between those values and the initial values of the drilling mud represents the change due to influx of formation fluids.

In addition to the partition coefficients, the analysis of the data from the gas analyzer can take into consideration the equation of continuity, shown below as Equation 1 (general equation) and Equation 2 (Cartesian coordinates):

$$\begin{matrix}[ \partial ρ / \partial t + ( ∇ \cdot ρ v ) = 0 ] & [ Eq . 1 ] \\ \frac{\partial ρ}{\partial t} + \frac{\partial }{\partial x} ( ρ {v}_{x} ) + \frac{\partial }{\partial y} ( ρ {v}_{y} ) + \frac{\partial }{\partial z} ( ρ {v}_{z} ) = 0 & [ Eq . 2 ]\end{matrix}$$

where ρ represents density and ν represents velocity.

The analysis also considers the equation of continuity for species a in terms of j_{α}, shown below as Equation 3 (general equation) and Equation 4 (Cartesian coordinates):

$$\begin{matrix} [ ρ D {ω}_{α} / Dt = - ( ∇ \cdot {j}_{α} ) + {r}_{α } ] & [ Eq . 3 ] \\ ρ ( \frac{\partial {ω}_{α}}{\partial t} + {v}_{x} \frac{\partial {ω}_{α}}{\partial x} + {v}_{y} \frac{\partial {ω}_{α}}{\partial y} + {v}_{z} \frac{\partial {ω}_{α}}{\partial z} ) = - [ \frac{\partial {j}_{α x}}{\partial x} + \frac{\partial {j}_{α y}}{\partial y} + \frac{\partial {j}_{α z}}{\partial z} ] + {r}_{α} & [ Eq . 4 ]\end{matrix}$$

where ρ represents density, ω_{α} represents mass fraction, j_{α} represents mass flux, and r_{α} represents mass rate of production by chemical reaction.

The analysis may also consider the equation of continuity for species A in terms of ω_{A} for constant ρδ_{AB}, shown below as Equation 5 (general equation) and Equation 6 (Cartesian coordinates).

$$\begin{matrix} [ ρ D {ω}_{A} / Dt = {ρδ}_{AB} {∇}^{2} {ω}_{A} + {r}_{A} ] & [ Eq . 5 ] \\ ρ ( \frac{\partial {ω}_{A}}{\partial t} + {v}_{x} \frac{\partial {ω}_{A}}{\partial x} + {v}_{y} \frac{\partial {ω}_{A}}{\partial y} + {v}_{z} \frac{\partial {ω}_{A}}{\partial z} ) = {ρδ}_{AB} [ \frac{{\partial }^{2} {ϖ}_{A}}{\partial {x}^{2}} + \frac{{\partial }^{2} {ϖ}_{A}}{\partial {y}^{2}} + \frac{{\partial }^{2} {ϖ}_{A}}{\partial {z}^{2}} ] + {r}_{A} & [ Eq . 6 ]\end{matrix}$$

where ρ represents density, ω represents mass fraction, δ represents Kronecker delta, and r represents mass rate of production by chemical reaction.

In certain embodiments, the analysis may also consider the equation of energy in terms of q, show below as Equation 7 (general equation) and Equation 8 (Cartesian coordinates).

$$\begin{matrix} [ ρ {\hat{C}}_{p} DT / Dt = - ( ∇ \cdot q ) - {( \partial ln ρ / \partial ln T )}_{ρ} D ρ / Dt - ( τ : ∇ v ) ] & [ Eq . 7 ] \\ ρ {\hat{C}}_{p} ( \frac{\partial T}{\partial t} + {v}_{x} \frac{\partial T}{\partial x} + {v}_{y} \frac{\partial T}{\partial y} + {v}_{z} \frac{\partial T}{\partial z} ) = - [ \frac{\partial {q}_{x}}{\partial x} + \frac{\partial {q}_{y}}{\partial y} + \frac{\partial {q}_{z}}{\partial z} ] - {( \frac{\partial ln ρ}{\partial ln T} )}_{ρ} \frac{D ρ}{Dt} - ( τ : ∇ v ) & [ Eq . 8 ]\end{matrix}$$

where ρ represents density, Ĉ represents heat capacity at constant pressure, qᵢ represents heat flux vector, T represents temperature, and τ represents momentum flux tensor.

Finally, the analysis may consider the equation of motion in terms of τ. These are illustrated below as Equation 9 (general equation) and Equations 10-12 (Cartesian coordinates).

$$\begin{matrix} [ ρ D v / Dt = - ∇ p - [ ∇ \cdot τ ] + ρ g ] & [ Eq . 9 ] \\ ρ ( \frac{\partial {v}_{x}}{\partial t} + {v}_{x} \frac{\partial {v}_{x}}{\partial x} + {v}_{y} \frac{\partial {v}_{x}}{\partial y} + {v}_{z} \frac{\partial {v}_{x}}{\partial z} ) = - \frac{\partial p}{\partial x} - [ \frac{\partial }{\partial x} {τ}_{xx} + \frac{\partial }{{\partial }_{y}} {τ}_{yx} + \frac{\partial }{\partial z} {τ}_{zx} ] + ρ {g}_{x} & [ Eq . 10 ] \\ ρ ( \frac{\partial {v}_{y}}{\partial t} + {v}_{x} \frac{\partial {v}_{y}}{\partial x} + {v}_{y} \frac{\partial {v}_{y}}{\partial y} + {v}_{z} \frac{\partial {v}_{y}}{\partial z} ) = - \frac{\partial p}{\partial y} - [ \frac{\partial }{\partial x} {τ}_{xy} + \frac{\partial }{{\partial }_{y}} {τ}_{yy} + \frac{\partial }{\partial z} {τ}_{zy} ] + ρ {g}_{y} & [ Eq . 11 ] \\ ρ ( \frac{\partial {v}_{z}}{\partial t} + {v}_{x} \frac{\partial {v}_{z}}{\partial x} + {v}_{y} \frac{\partial {v}_{z}}{\partial y} + {v}_{z} \frac{\partial {v}_{z}}{\partial } ) = - \frac{\partial p}{\partial z} - [ \frac{\partial }{\partial x} {τ}_{xz} + \frac{\partial }{{\partial }_{y}} {τ}_{yz} + \frac{\partial }{\partial z} {τ}_{zz} ] + ρ {g}_{z} & [ Eq . 12 ]\end{matrix}$$

where ρ represents density, νᵢ represents velocity, g represents gravity, and τᵢⱼ represents momentum flux tensor.

A person of ordinary skill would recognize that these equations can be used with the Cartesian coordinate system, as is shown above, or that cylindrical and spherical coordinates may be used as well. Moreover, a person of ordinary skill in the art with the benefit of this disclosure would be able to adopt these equations to reflect the appropriate characteristics and boundary values of the reservoir.

In certain embodiments, the gas extractor uses constant temperature, pressure, and flow. This includes, for example, the EAGLE system. Embodiments that use such a gas extractor can also include the appropriate heaters and flow control valves to ensure that the gas extractor's input stream remains constant. In other embodiments, the gas extractor can use variable flow and variable pressure. Examples of these embodiments include Texaco Trap and QGM systems. In these embodiments, it is important for the analysis to take into consideration the potential change of pressure and flow rate.

These measurements and equations are used to create a model of the mud's equilibrium. In different embodiments of the present disclosure, the model can use mass ratios or molar ratios. By using the model of the mud's equilibrium, a person of skill in the art may calculate the amount of any component in the mud (e.g., hydrocarbons) based the measurement of other components. A person of skill in the art may also compare expected concentrations of a component (overall or in each phase) against actual measurements in order to provide information about the composition of the subterranean reservoir.

This analysis may be conducted at selected (e.g., predetermined) points in time during an operation, or may be performed continuously throughout the drilling operation. In some embodiments, the analysis described above is performed in real-time. In some embodiments, some or all of the data may be transmitted to an offsite location, for example, where wells at one or more sites may be monitored by the same personnel substantially simultaneously. This permits more efficient monitoring of the drilling operations because the appropriate personnel may be centrally located. In certain embodiments, data from the gas analyzer is automatically uploaded into a central database and acquisition system.

The present invention is therefore well-adapted to carry out the objects and attain the ends mentioned, as well as those that are inherent therein. While the invention has been depicted and described by references to examples of the invention, such a reference does not imply a limitation on the invention, and no such limitation is to be inferred. The invention is capable of considerable modification, alteration and equivalents in form and function, as will occur to those ordinarily skilled in the art having the benefit of this disclosure. The depicted and described examples are not exhaustive. Consequently, the invention is intended to be limited only by the spirit and scope of the appended claims, giving full cognizance to equivalents in all respects.

An embodiment of the present disclosure is a method comprising: exposing a fluid to at least a portion of a subterranean reservoir; returning the fluid to the surface; removing a gas sample from the fluid after the fluid has been returned to the surface; analyzing the gas sample to determine at least one characteristic of the gas sample; and using the characteristic of the gas sample and at least one known parameter of the fluid to calculate at least one characteristic of at least one non-gas phase of the fluid. Optionally, the gas sample is analyzed using a hydrocarbon analyzer. Optionally, the gas sample is analyzed using gas spectroscopy. Optionally, the characteristic of the gas sample comprises the concentration of a component of the gas sample. Optionally, the known parameters comprise partitioning coefficients. Optionally, the characteristic of the gas sample is transmitted to an offsite location. Optionally, the characteristic of a non-gas phase of the fluid is calculated at an offsite location. Optionally, the method further comprises determining at least one initial characteristic of the gas phase of the fluid before the fluid has been exposed to the subterranean reservoir. Optionally, the method further comprises comparing the initial characteristic of the gas phase of the fluid to the characteristic of the gas sample. Optionally, the method further comprises transmitting data regarding the characteristic of the gas sample to an offsite location.

Another embodiment of the present disclosure is a method comprising: determining at least one initial characteristic of the gas phase of a drilling fluid; circulating the drilling fluid in a subterranean reservoir where the drilling fluid is exposed to a reservoir fluid and then returned to the surface; removing a gas sample from the drilling fluid after the drilling fluid has been returned to the surface; analyzing the gas sample to determine at least one characteristic of the gas sample; using the characteristic of the gas sample and at least one known parameter of the drilling fluid to calculate at least one characteristic of at least one non-gas phase of the drilling fluid; and comparing the initial characteristic of the gas phase of the drilling fluid to the characteristic of the gas sample.

Another embodiment of the present disclosure is a system comprising: a gas extractor that removes a gas sample from a fluid after the fluid has been exposed to at least a portion of a subterranean reservoir and then returned to the surface; a gas analyzer that analyzes the gas sample to determine at least one characteristic of the gas sample; and a controller that uses the characteristic of the gas sample and at least one known parameter of the fluid to calculate at least one characteristic of at least one non-gas phase of the fluid. Optionally, the gas analyzer comprises a hydrocarbon analyzer. Optionally, the characteristic of the gas sample comprises the concentration of a component of the gas sample. Optionally, the known parameters comprise partitioning coefficients. Optionally, the system further comprises an offsite location that receives a transmission containing the characteristic of the gas sample. Optionally, the controller is located at the offsite location.

Another embodiment of the present disclosure is a system comprising: a gas extractor that removes a gas sample from a fluid after the fluid has been exposed to at least a portion of a subterranean reservoir and then returned to the surface; a gas analyzer that analyzes the gas sample to determine at least one characteristic of the gas sample; a central database and acquisition system that uploads data from the gas analyzer; and a controller that uses the characteristic of the gas sample and at least one known parameter of the fluid to calculate at least one characteristic of at least one non-gas phase of the fluid.

What is claimed is:

1. A method comprising:
exposing a fluid to at least a portion of a subterranean reservoir;
returning the fluid to the surface;
removing a gas sample from the fluid after the fluid has been returned to the surface;
analyzing the gas sample to determine at least one characteristic of the gas sample; and
using the characteristic of the gas sample and at least one partitioning coefficient to calculate at least one characteristic of at least one non-gas phase of the fluid.

2. The method of claim 1 wherein the gas sample is analyzed using a hydrocarbon analyzer.

3. The method of claim 1 wherein the gas sample is analyzed using gas spectroscopy.

4. The method of claim 1 wherein the characteristic of the gas sample comprises the concentration of a component of the gas sample.

5. (canceled)

6. The method of claim 1 wherein the characteristic of the gas sample is transmitted to an offsite location.

7. The method of claim 6 wherein the characteristic of a non-gas phase of the fluid is calculated at an offsite location.

8. The method of claim 1 further comprising the step of:
determining at least one initial characteristic of the gas phase of the fluid before the fluid has been exposed to the subterranean reservoir.

9. The method of claim 8 further comprising the step of:
comparing the initial characteristic of the gas phase of the fluid to the characteristic of the gas sample.

10. The method of claim 1 further comprising transmitting data regarding the characteristic of the gas sample to an offsite location.

11. A method comprising:
determining at least one initial characteristic of the gas phase of a drilling fluid;
circulating the drilling fluid in a subterranean reservoir where the drilling fluid is exposed to a reservoir fluid and then returned to the surface;
removing a gas sample from the drilling fluid after the drilling fluid has been returned to the surface;
analyzing the gas sample to determine at least one characteristic of the gas sample;
using the characteristic of the gas sample and at least one partitioning coefficient to calculate at least one characteristic of at least one non-gas phase of the drilling fluid; and
comparing the initial characteristic of the gas phase of the drilling fluid to the characteristic of the gas sample.

12. A system comprising:
a gas extractor that removes a gas sample from a fluid after the fluid has been exposed to at least a portion of a subterranean reservoir and then returned to the surface;
a gas analyzer that analyzes the gas sample to determine at least one characteristic of the gas sample; and
a controller that uses the characteristic of the gas sample and at least one partitioning coefficient to calculate at least one characteristic of at least one non-gas phase of the fluid.

13. The system of claim 12 wherein the gas analyzer comprises a hydrocarbon analyzer.

14. The system of claim 12 wherein the characteristic of the gas sample comprises the concentration of a component of the gas sample.

15. (canceled)

16. The system of claim 12 further comprising an offsite location that receives a transmission containing the characteristic of the gas sample.

17. The system of claim 16 wherein the controller is located at the offsite location.

18. A system comprising:
a gas extractor that removes a gas sample from a fluid after the fluid has been exposed to at least a portion of a subterranean reservoir and then returned to the surface;
a gas analyzer that analyzes the gas sample to determine at least one characteristic of the gas sample;
a central database and acquisition system that uploads data from the gas analyzer; and
a controller that uses the characteristic of the gas sample and at least one partitioning coefficient to calculate at least one characteristic of at least one non-gas phase of the fluid.
